(19) United States
(12) Patent Application Publication
(10) Pub. No.: US 2009/0324573 A1
(43) Pub. Date: Dec. 31, 2009

(54) Pharmaceutical Composition Comprising P43 Protein for the Treatment of Gastric Adenocarcinoma

(76) Inventors: Datao Liu, Shanghai (CN); Xiaoding Tan, Shanghai (CN); Xi Zhu, Shanghai (CN); Yan Wang, Shanghai (CN); Linda Hu, Shanghai (CN)

Correspondence Address: HAMRE, SCHUMANN, MUELLER & LARSON, P.C., P.O. BOX 2902, MINNEAPOLIS, MN, 55402-0902, US

(21) Appl. No.: 12/472,042

(22) Filed: May 26, 2009

Related U.S. Application Data
(63) Continuation of application No. PCT/CN2007/070965, filed on Oct. 26, 2007.

(30) Foreign Application Priority Data
Nov. 29, 2006 (CN) 200610118859.8

(51) Int. Cl. A61K 38/45

(52) U.S. Cl. 424/94.5

(57) ABSTRACT

A pharmaceutical composition for the treatment of gastric adenocarcinoma is provided, wherein the pharmaceutical composition comprises p43 protein and pharmaceutically acceptable carriers, and does not comprise paclitaxel. A use of p43 protein for the treatment of gastric adenocarcinoma is also provided.

### TECHNICAL FIELD

The present invention relates to a pharmaceutical composition for treatment of gastric adenocarcinoma, in particular to a pharmaceutical composition for treatment of gastric adenocarcinoma and its use in treatment of gastric adenocarcinoma.

### BACKGROUND ART

Malignant tumor is very harmful to health of human being. The methods for treating malignant tumor in the medical field mainly comprise killing cancer cell via surgery, chemotherapy and teletherapy, and the like. These methods may kill normal cell when killing cancer cell, and they may readily make the cancer cell producing drug resistance. Therefore, people has all the while found a method that could specifically kill cancer cell without damaging normal cell, and at the same time did not make the cancer cell producing drug resistance. Currently, there emerge in endlessly a lot of new drugs for treating tumor. Recently, people gradually focuses on the methods for treating malignant tumor by genetic engineering drug, with development of molecular biology gene engineering. Conventional chemotherapy for treating tumor uses drugs having cytotoxicity which may damage not only cancer cells but also normal cells, thereby weakening human body condition. So, many patients of advanced cancer finally die because they are unable to endure the treatment. Malignant tumors have one common feature, i.e., cells in a local area greatly and un-controllably proliferate. In order to maintain this cell proliferation, it needs to provide much nutrition. When forming tumor; many blood vessels for supplying nutrition components are formed within and around the tumor. If the “channels” for supplying are cut to interrupt the nutrition components supply for the tumor, the tumor will die due to depletion.

A method of inhibiting neovascularization is recently developed for completely treating cancer. It is found that the growth of tumor especially solid tumor needs much nutrition supply. And many neogenic vessels are formed near the tumor. The method of inhibiting neovascularization may stop the formation of the blood vessels in tumor mainly through inhibiting the growth of vascular endothelial cell that fast and abnormally proliferate, thereby specially inhibiting the growth of proliferating vascular endothelial cells in tumor. It atrophies capillary vessel of tumor, so the nutrition supply of tumor is cut and the cancer cell is dead. The object of treating cancer is thus achieved.

Upon continuous study of about ten years, a plurality of potential inhibitors for neovascularization have been found, including human endostatin. It was firstly reported in Cell, January 1997 about Endostatin by Folk-man from Medical College, Harvard, finding that two drugs (Angiostatin and Endostatin) may remove tumor in rat without recurrence. These two drugs can treat cancer by stopping the growth of blood vessels which supply nutrition components for tumor. This made a big splash at that time. Endostatin was rapidly studied in the field of cancer treatment. In the beginning of 1999, at that time the clinical studies were not completed, FDA specifically admitted clinical trial for using a mixed gene engineering injection of Angiostatin and Endostatin for treating 30 patients with advanced cancer which were non-responded to the other drugs. Experts in American Society of Clinical Oncology believe that these drugs may be successful. Now, Endostatin has passed the first clinical trial and it entered into the second clinical trial. However, since Endostatin has poor solubility, resulting in high cost of production. Meanwhile, it is used only in aqueous injection dosage form, which greatly limits its applications.

p43 protein is an accessory factor of tRNA synthetase of mammals, which on one hand adjusts directly the formation of capillary vessel from endothelial cells, and on the other hand inhibits the formation of blood vessel of tumor by changing microenvironment. The activity of p43 in inhibiting formation of blood vessel and growth of tumor of protein have been proved by in vitro tests and animal tests. Human gene recombinant p43 protein shows the potential of being developed into a new drug for treating cancer. It may be effective in treating a plurality of primary and metastatic solid tumors and may be used along with chemotherapy and teletherapy.

p43 protein is a member of human aminoacyl-tRNA synthetase and a precursor of endothelial monocyte active peptide, which have been found in 1997 by Sophie Q, et al. p43 protein is a single-chain protein, comprising 312 amino acids totally and its secondary structure includes 11 β-sheets. Imagene Company in South Korea have thoroughly studied the structure and activity of human p43 protein, finding that p43 protein may inhibit the growth of vascular endothelial cell that proliferate fast and abnormally and the growth of neogenic vessels in hen'egg test-chorioallantoic membrane, which means that p43 protein may have a potential of treating cancer. However, it is uncertain to which solid tumor this protein may be effective, that is to say, it is unknown about the antitumor spectrum of p43 protein.

No specific drug for treating gastric adenocarcinoma were provided in the art. Therefore, there is a need for a specific pharmaceutical composition for treating gastric adenocarcinoma.

### SUMMARY OF THE INVENTION

The object of the present invention is to provide a pharmaceutical composition for effectively treating gastric adenocarcinoma.

The present invention provides a pharmaceutical composition for treating gastric adenocarcinoma, comprising p43 protein and pharmaceutically acceptable carriers, wherein the pharmaceutical composition does not include paclitaxel.

In one preferred embodiment, the amount of p43 protein is from 0.1% to 99.9%.

In one preferred embodiment, the amount of p43 protein is from 5% by weight to 95% by weight.

In one preferred embodiment, the amount of p43 protein is from 10% by weight to 60% by weight.

In one preferred embodiment, the amount of p43 protein is from 20% by weight to 40% by weight.

In one preferred embodiment, the pharmaceutically acceptable carrier is selected from physiological saline.

In one preferred embodiment, the pharmaceutical composition further comprises human albumin.

In one preferred embodiment, the amount of the human albumin is from 0.01% by weight to 10% by weight.

The present invention further provides use of p43 protein in preparation of a medicament for treating gastric adenocarcinoma.

The pharmaceutical composition according to the present invention can specifically treat gastric adenocarcinoma, without any side effect.

### BRIEF DESCRIPTION OF THE FIGURES

FIG. 1 describes the effect of p43 protein against human gastric adenocarcinoma.

### DETAILED DESCRIPTION OF THE INVENTION

The present invention provides a pharmaceutical composition for treating gastric adenocarcinoma, comprising p43 protein and pharmaceutically acceptable carriers, wherein the pharmaceutical composition does not include paclitaxel.

The p43 protein is conventional in the art, and its preparation and sequence have been disclosed in the art, see U.S. Pat. No. 5,641,867 and WO0195927, all of the contents of which are hereby incorporated by reference. In one preferred embodiment, the p43 protein is provided by Sine Laboratory, Shanghai, China.

In the pharmaceutical composition according to the present invention, the amount of p43 protein is conventional, and a skilled person in the art can directly determine its amount according to the prior art. In one preferred embodiment, the amount of p43 protein is from 0.1% by weight to 99.9% by weight, preferably from 5% by weight to 95% by weight, more preferably from 10% by weight to 60% by weight, and most preferably from 200%) by weight to 40% by weight.

In the present invention, the pharmaceutically acceptable carrier is conventional, and a skilled person in the art can determine directly which pharmaceutically acceptable carrier can be used in the present invention based on the prior art. In one preferred embodiment, the pharmaceutically acceptable carrier is selected from physiological saline. The amount of the pharmaceutically acceptable carrier is generally from 0.1% by weight to 99.9% by weight, preferably from 5% by weight to 95% by weight, more preferably from 40% by weight to 90% by weight, and most preferably from 60% by weight to 80% by weight.

The pharmaceutical composition according to the present invention may further comprise other agents, such as human albumin. In one preferred embodiment, the amount of human albumin is from 0.01% by weight to 10% by weight, preferably from 0.1% by weight to 5% by weight, more preferably from 0.2% by weight to 1% by weight, and most preferably 0.3% by weight.

The pharmaceutical composition according to the present invention generally contains no paclitaxel.

The pharmaceutical composition may be produced to any suitable dosage form, such as oral liquid, tablet, capsule, oral disintegrating tablet, and injection liquid and the like. A skilled person in the art can easily produce the above pharmaceutical composition into the above dosage forms according to the prior art.

The amount of p43 protein is conventional, and a skilled person in the art can determine directly its amount based on the prior art. Generally, the amount of p43 protein is from 1 to 10 mg/kg/day, preferably from 2 to 8 mg/kg/day, more preferably from 3 to 7 mg/kg/day, and most preferably 5 mg/kg/day.

The present invention on the other hand further provides use of p43 protein in preparation of medicament for treating gastric adenocarcinoma.

The present invention is described by reference to the following examples, but are not limited hereto.

### EXAMPLES

### Example 1

### Animal

Animal Resource, Line and Strain: BALB/cA bare mouse, provided by Experimental Animal Center, Chinese Academy of Sciences, Shanghai, China.

Certificate No.: 122 (HU)

Age: 35-40 days

Weight: 18-22 g

Gender: Male & Female

Transplanted Tumor

Bare mouse transplanted with human gastric adenocarcinoma SGC-71901 was established by subcutaneously inoculating human gastric adenocarcinoma SGC-7901 cell strain into bare mouse, with a inoculation concentration of 1-3×10⁶. After establishing the transplanted tumor, it was passaged in bare mouse for three generations prior to use.

Experiments

The eugonic tumor tissue was cut into about 1.5 mm³. It was inoculated into hypodermis in right armpit of bare mouse under aseptic condition. The diameter of the transplanted tumor of the bare mouse was measured by vernier caliper. After the tumor grew to 100-300 mm³, the mice were grouped randomly. The effect of reagents used for inhibiting tumor was observed dynamically through measuring the diameter of tumor. The diameter of tumor was measured 3 times per week, while measuring the weight of mice. With respect to the mice in an administration group, the mice were administrated by intravenous injection 6 times per week: with respect to the mice in a positive control group, the mice were administrated by intravenous injection 2 times per week; with respect to the mice in a negative control group, the mice were administrated by same amount of physiological saline at the same time.

The tumor volume (TV) was calculated as follows:

TV=½×a×b²

in which a and b represent length and width, respectively. The relative tumor volume (RTV) was calculated based on the measured results: RTV=Vₜ/V₀, in which V₀ represents the tumor volume measured at the first administration (d0), and Vₜ represents the tumor volume measured at each measurement. The factor for estimating the effect against tumor is the relative tumor proliferation T/C (%) as follows:

T/C(%)=T_{RTV}/C_{RTV}×100

T_{RTV}: RTV for treating group; C_{RTV}: RTV for negative control group.

100 mg of p43 protein, obtained from Sine Laboratory in Shanghai, China, was dissolved in physiological saline to form a solution of 1 mg/ml.

The solution was administrated to bare mouse by intravenous injection in 2.5 mg p43 protein/kg weight, 5 mg p43 protein/kg weight, and 10 mg p43 protein/kg weight. The results were listed in the following Table 1.

TABLE 1

| | Dosage | Number of Animals | | Weight (g) | | TV | | | T/C |
|---|---|---|---|---|---|---|---|---|---|
| Group | mg/kg | First | Final | First | Final | d 0 | d 21 | RTV | (%) |
| physiological | / | 10 | 10 | 20.4 ± 1.1 | 24.9 ± 2.3 | 143 ± 63 | 3011 ± 421 | 20.98 ± 4.05 | / |
| saline | | | | | | | | | |
| P43 | 10 | 5 | 5 | 20.7 ± 0.6 | 25.4 ± 2.8 | 132 ± 34 | 1715 ± 168 | 12.99 ± 5.14 | 61.9 |
| P43 | 5 | 5 | 5 | 20.5 ± 0.7 | 25.1 ± 1.9 | 146 ± 72 | 1648 ± 102 | 11.37 ± 6.3 | 54.2 |
| P43 | 2.5 | 5 | 5 | 20.3 ± 0.9 | 26.7 ± 1.7 | 140 ± 73 | 2400 ± 360 | 17.14 ± 7.25 | 81.7 |

d 0: the day beginning the first administration
d 21: the twenty-first day after the first administration

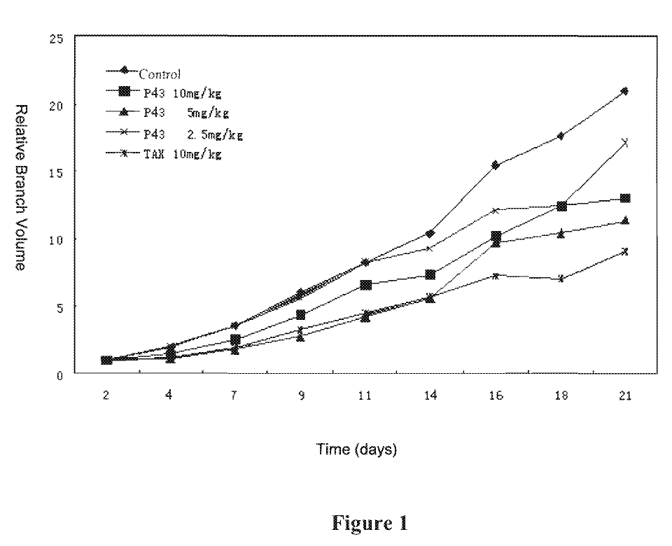

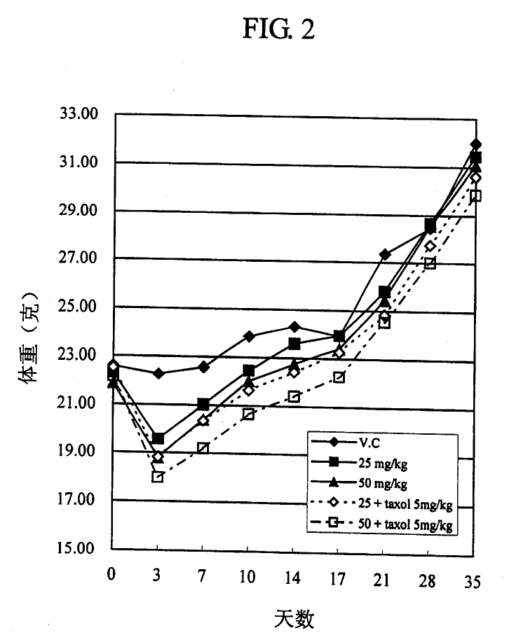

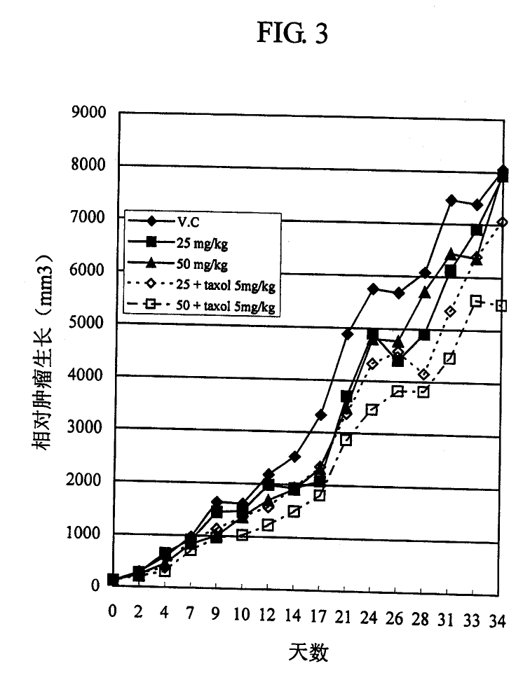

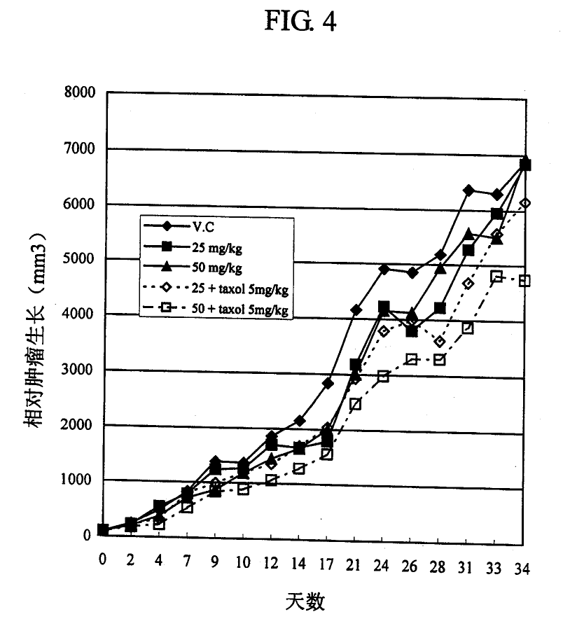

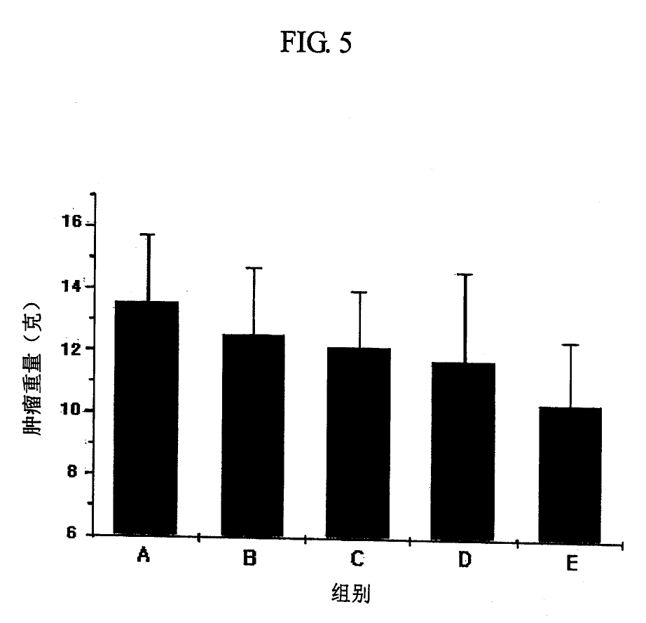

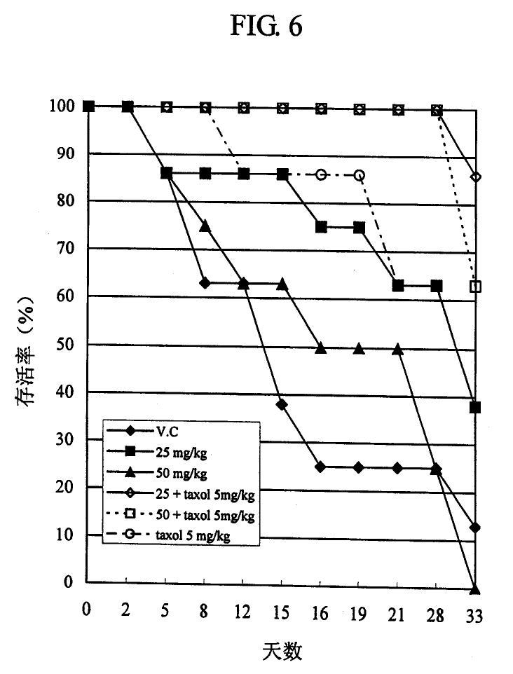

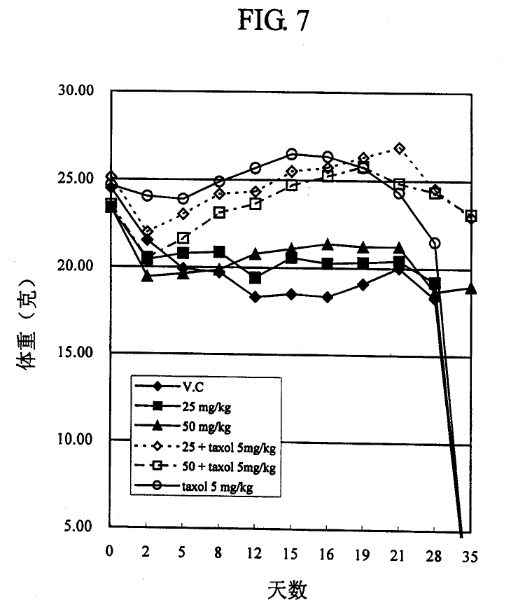

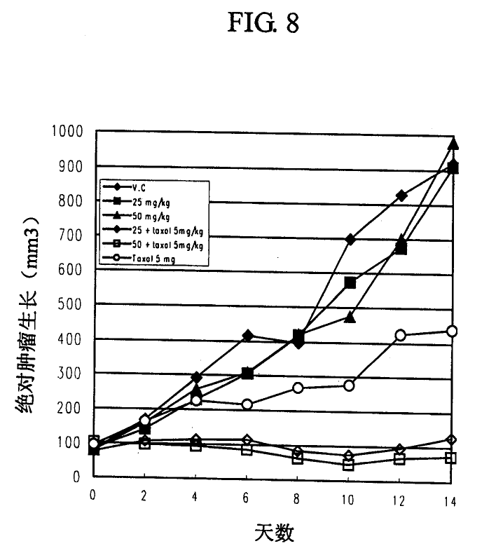

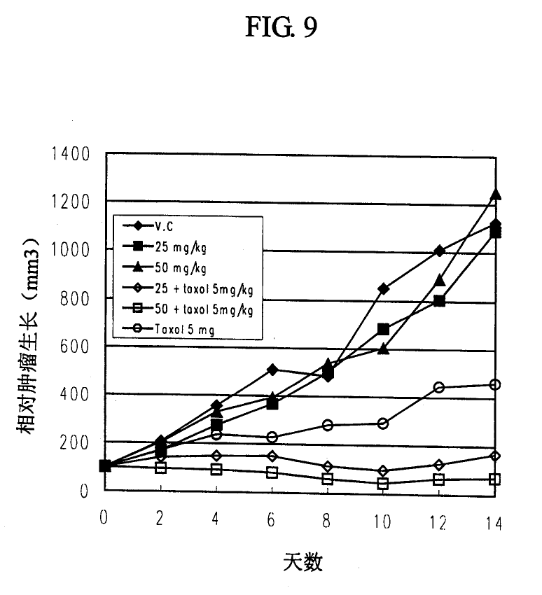

From the above table, the effect of p43 protein against human gastric adenocarcinoma SGC-7901 can be seen from Table 1 and FIG. 1. p43 protein has significant inhibition on growth of transplanted tumor of human gastric adenocarcinoma SGC-7901 in bare mouse. The T/C (%) of p43 2.5 mg/kg, 5 mg/kg and 10 mg/kg against SGC-7901 was 81.7, 54.2 and 61.9, respectively, without any obvious side effect.

### Example 2

### Animal

Animal Resource, Line and Strain: BALB/cA bare mouse, provided by Experimental Animal Center, Chinese Academy of Sciences, Shanghai, China.

Certificate No.: 122 (HU)

Age: 35-40 days

Weight: 18-22 g

Gender: Male & Female

Number of animal in each group: 10 mice for negative control group and 5 mice for treating group

Transplanted Tumor

Transplanted tumor of human colon carcinoma HCT-116 in bare mouse was established by inoculating human colon carcinoma HCT-116 cell strain into hypoderm of bare mouse, with a inoculation concentration of 1-3×10⁶. After establishing the transplanted tumor, it was passaged in bare mouse for three generations prior to use.

Experiments

The eugonic tumor tissue was cut into about 1.5 mm³. It was inoculated into hypodermis in right armpit of bare mouse under aseptic condition. The diameter of the transplanted tumor of the bare mouse was measured by vernier caliper. After the tumor grew to 100-300 mm³, the mice were grouped randomly. The effect of reagents used for inhibiting tumor was observed dynamically through the method of measuring the diameter of tumor. The diameter of tumor was measured 3 times per week, while measuring the weight of mice. With respect to the mice in an administration group, the mice were administrated by intravenous injection 6 times per week, with respect to the mice in a positive control group, the mice were administrated by intravenous injection 2 times per week; with respect to the mice in a negative control group, the mice were administrated by same amount of physiological saline at the same time.

The tumor volume (TV) was calculated as follows:

TV=½×a×b²

in which a and b represent length and width, respectively. The relative tumor volume (RTV) was calculated depending on the measured results: RTV=Vₜ/V₀, in which V₀ represents the tumor volume measured at the first administration (d0), and Vₜ represents the tumor volume measured at each measurement. The factor for estimating the effect against tumor is the relative tumor growth T/C (%) as follows:

T/C(%)=T_{RTV}/C_{RTV}×100

T_{RTV}: RTV for treating group; C_{RTV}: RTV for negative control group.

100 mg of p43 protein, available from Sine Laboratory in Shanghai, China, was dissolved in physiological saline to form a solution of 1 mg/ml.

The solution was administrated to bare mouse by intravenous injection in 2.5 mg p43 protein/kg weight, 5 mg p43 protein/kg weight, and 10 mg p43 protein/kg weight. The results were listed in the following Table 2.

TABLE 2

| | Dosage | Number of animal | | Weight (g) | | TV | | | T/C |
|---|---|---|---|---|---|---|---|---|---|
| Group | mg/kg | First | Final | First | Final | d 0 | d 21 | RTV | (%) |
| physiological | / | 10 | 10 | 20.2± | 23.6 ± 1.8 | 165 ± 44 | 978 ± 274 | 5.93 ± 0.82 | / |
| saline | | | | | | | | | |
| P43 | 10 | 5 | 5 | 20.9± | 23.5 ± 2.1 | 161 ± 52 | 761 ± 211 | 4.73 ± 1.01 | 79.7 |
| P43 | 5 | 5 | 5 | 21.3± | 23.9 ± 2.2 | 156 ± 39 | 703 ± 189 | 4.51 ± 0.75 | 76.0 |
| P43 | 2.5 | 5 | 5 | 20.7± | 24.1 ± 1.5 | 164 ± 48 | 822 ± 145 | 5.01 ± 0.92 | 84.5 |

d 0: the day beginning the first administration
d 21: the twenty-first day after the first administration

The effect of p43 protein against human colon carcinoma HCT-116 can be seen from Table 2. p43 protein has non-significant inhibition on growth or transplanted tumor of human colon carcinoma HCT-116 in bare mouse. The T/C (%) of p43 2.5 mg/kg, 5 mg/kg and 10 mg/kg against HCT-116 was 84.5, 76.0 and 79.79 respectively, without any obvious side effect.

### Example 3

### Animal

Animal Resource, Line and Strain: BALB/cA bare mouse, provided by Experimental Animal Center, Chinese Academy of Sciences, Shanghai, China.

Certificate No.: 122 (HU)

Age: 35-40 days

Weight: 18-22 g

Gender: Male & Female

Number of animal in each group: 10 mice for negative control group and 5 mice for treating group

Transplanted Tumor

Transplanted tumor of human nasopharyngeal carcinoma CNE-1 in bare mouse was established by inoculating human nasopharyngeal carcinoma CNE-1 cell strain into hypoderm of bare mouse, with a inoculation concentration of 1-3×10⁶. After establishing the transplanted tumor, it was passaged in bare mouse for three generations prior to use.

Experiments

The eugonic tumor tissue was cut into about 1.5 mm³. It was inoculated into hypodermis in right armpit of bare mouse under aseptic condition. The diameter of the transplanted tumor of the bare mouse was measured by vernier caliper. After the tumor grew to 100-300 mm³, the mice were grouped randomly. The effect of reagents used fro inhibiting tumor was observed dynamically through the method of measuring the diameter of tumor. The diameter of tumor was measured 3 times per week, while measuring the weight of mice. With respect to the mice in an administration group, the mice were administrated by intravenous injection 6 times per week; with respect to the mice in a positive control group, the mice were administrated by intravenous injection 2 times per week; with respect to the mice in a negative control group, the mice were administrated by same amount of physiological saline at the same time.

The tumor volume (TV) was calculated as follows:

TV=½×a×b²

in which a and b represent length and width, respectively. The relative tumor volume (RTV) was calculated depending on the measured results: RTV=Vₜ/V₀, in which V₀ represents the tumor volume measured at the first administration (d0), and Vₜ represents the tumor volume measured at each measurement. The factor for estimating the effect against tumor is the relative tumor growth T/C (%) as follows:

T/C(%)=T_{RTV}/C_{RTV}×100

T_{RTV}: RTV for treating group; C_{RTV}: RTV for negative control group.

100 mg of p43 protein, available from Sine Laboratory in Shanghai, China, was dissolved in physiological saline to form a solution of 1 mg/ml.

The solution was administrated to bare mouse by intravenous injection in 2.5 mg p43 protein/kg weight, 10 mg p43 protein/kg weight, and 10 mg p43 protein/kg weight. The results were listed in the following Table 3.

TABLE 3

| | Dosage | Number of animal | | Weight (g) | | TV | | | T/C |
|---|---|---|---|---|---|---|---|---|---|
| Group | mg/kg | First | Final | First | Final | d 0 | d 21 | RTV | (%) |
| physiological | / | 10 | 10 | 20.2 ± 0.6 | 24.3 ± 1.6 | 141 ± 50 | 2130 ± 438 | 15.11 ± 6.83 | / |
| saline | | | | | | | | | |
| P43 | 10 | 5 | 5 | 19.8 ± 1.1 | 22.9 ± 2.3 | 147 ± 63 | 1856 ± 173 | 12.63 ± 4.88 | 83.6 |
| P43 | 5 | 5 | 5 | 19.6 ± 0.8 | 23.1 ± 1.8 | 134 ± 48 | 1708 ± 154 | 12.75 ± 5.12 | 84.4 |
| P43 | 2.5 | 5 | 5 | 20.5 ± 1.3 | 24.6 ± 2.4 | 143 ± 70 | 1921 ± 201 | 13.43 ± 5.64 | 88.9 |

d 0: the day beginning the first administration
d 21: the twenty-first day after the first administration

The effect of p43 protein against human nasopharyngeal carcinoma CNE-1 can be seen from Table 3. p43 protein has non-significant inhibition on growth of transplanted tumor of human nasopharyngeal carcinoma CNE-1 in bare mouse. The T/C (%) of p43 2.5 mg/kg, 5 mg-kg and 10 mg/kg against CNE-1 was 88.9, 84.4 and 83.6, respectively, without any obvious side effect.

What is claimed is:

1. A pharmaceutical composition for treating gastric adenocarcinoma, comprising p43 protein and pharmaceutically acceptable carriers, wherein the pharmaceutical composition does not include paclitaxel.

2. The pharmaceutical composition according to claim 1, wherein the amount of p43 protein is from 0.1% to 99.9%.

3. The pharmaceutical composition according to claim 1, wherein the amount of p43 protein is from 5% by weight to 95% by weight.

4. The pharmaceutical composition according to claim 1, wherein the amount of p43 protein is from 10% by weight to 60% by weight.

5. The pharmaceutical composition according to claim 1, wherein the amount of p43 protein is from 20% by weight to 40% by weight.

6. The pharmaceutical composition according to claim 1, wherein the pharmaceutically acceptable carrier is selected from physiological saline.

7. The pharmaceutical composition according to claim 1, wherein the pharmaceutical composition further comprises human albumin.

8. The pharmaceutical composition according to claim 7, wherein the amount of the human albumin is from 0.01% by weight to 10% by weight.

9. Use of p43 protein in preparation of a medicament for treating gastric adenocarcinoma.
